(19) United States
(12) Patent Application Publication
(10) Pub. No.: US 2005/0043231 A1
(43) Pub. Date: Feb. 24, 2005

(54) Therapy for growth hormone induced insulin resistance in juveniles with growth disorders

(76) Inventors: Wayne Stephen Cutfield, Auckland (NZ); Paul Leslie Hofman, Auckland (NZ); Bernhard Hermann Heinrich Breier, Auckland (NZ)

Correspondence Address: FLIESLER MEYER, LLP, FOUR EMBARCADERO CENTER, SUITE 400, SAN FRANCISCO, CA, 94111, US

(21) Appl. No.: 10/499,902

(22) PCT Filed: Dec. 23, 2002
(86) PCT No.: PCT/NZ02/00292

(30) Foreign Application Priority Data
Dec. 24, 2001 (NZ) 516421
Oct. 4, 2002 (NZ) 521824

(51) Int. Cl. A61K 38/27; A61K 31/4439; A61K 31/426; A61K 31/155

(52) U.S. Cl. 514/12; 514/342; 514/369; 514/635

(57) ABSTRACT

This invention provides compositions and methods for treating adverse effects of growth hormone therapy in animals that are small for gestational age or suffered from intrauterine growth restriction. Methods include providing growth hormone to increase growth rates, and includes co-administration of an insulin sensitiser, such as a biguanide or a thiazolidinedione. In other embodiments, combinations of insulin sensitisers can be co-administered along with growth hormone.

### TECHNICAL FIELD

The invention relates to a method and a composition to treat growth hormone treatment induced insulin resistance in juveniles with growth disorders. In particular, the invention relates to the treatment of growth hormone induced insulin resistance in juveniles with growth disorders in which pre-existing insulin resistance is a feature.

### BACKGROUND

Children born small for gestational age (SGA) are characterised by poor post-natal growth and short stature. SGA may result from: prematurity, growth hormone insufficiency, growth hormone deficiency or a condition known as Intrauterine Growth Retardation/Restriction (IUGR). IUGR describes a phenotype of restricted or retarded growth which is a consequence of adverse intrauterine environmental or genetic factors. These include maternal factors (e.g. multiple pregnancy, nutrition, placental abnormalities, or diseases) and fetal factors (e.g. chromosomal abnormalities, genetic abnormalities, skeletal development abnormalities, etc.).

The use of recombinant technology in production of GH has allowed for far greater quantities of GH to be available for therapeutic use. Consequently, GH therapy is no longer restricted for use in short children with GH deficiency. Growth hormone (GH) therapy is currently the main form of treatment administered to SGA children. Recombinant human growth hormone (rhGH) therapy has been shown to improve not only short term, but also long term height gain in short IUGR children and those affected by Turner Syndrome (Job et al. 1996, De Zegher et al. 1996, Azcona et al. 1998, Fjellestad et al. 1998, De Zegher et al. 2000, Rosenfeld et al. 1998, Ranke et al. 1996). High dose rhGH regimens have produced the greatest gains in height in these children (De Zegher et al. 1996, De Zegher et al. 2000).

The beneficial outcomes of rhGH therapy are not free from side-effects. Conflicting findings were reported on the changes in insulin sensitivity with short normal children during rhGH therapy. Heptula et al. demonstrated a reduction in insulin sensitivity with hyperglycaemic clamping during six months of rhGH therapy (Heptula et al. 1997). Conversely, Walker did not detect a reduction in insulin sensitivity during euglycemic clamping in short normal children who received one year of high dose rhGH treatment (Walker et al. 1989). However, these children were hyperinsulinemic during an oral glucose tolerance test, suggesting a reduction in insulin sensitivity (Walker et al. 1989).

Pre-existing insulin resistance may be a feature in children with certain growth disorders. The inventors have previously demonstrated that short children born SGA had reduced insulin sensitivity when matched to short normal children (Hofman et al. 1997). In girls with Turner Syndrome the trends in insulin sensitivity prior to and during rhGH therapy parallel inventors' findings in SGA children. Turner Syndrome girls exhibited reduced insulin sensitivity without rhGH therapy, with a further reduction in insulin sensitivity with rhGH therapy (Caprio et al. 1992). So far there has been no formal assessment of insulin sensitivity following rhGH therapy in children.

In the light of the above observations it is clearly advantageous to establish a method of eliminating or at least alleviating the side-effects of the GH treatment of childhood growth disorders.

### SUMMARY OF THE INVENTION

In certain embodiments of this invention, we disclose methods of prophylaxis and/or treatment of growth hormone induced insulin resistance or hyperinsulinemia in juveniles, comprising administering an effective amount of growth hormone in combination with an insulin sensitiser or a combination of insulin sensitisers.

In other embodiments of this invention, we disclose methods of treating growth hormone disorders in juveniles, the method comprising administering an effective amount of growth hormone in combination with an insulin sensitiser or a combination of insulin sensitisers.

In additional aspects of this invention, we disclose compositions suitable for treating growth hormone-induced insulin resistance.

Some of these embodiments are based on the surprising observation that insulin sensitizers can diminish or ameliorate adverse effects of growth hormone on insulin sensitivity in children with short stature.

### FIGURES

This invention is described with respect to certain embodiments thereof. Other features of embodiments of this invention are described in the figures, in which:

FIG. 1 depicts a graph showing changes in insulin sensitivity prior to, during and following GH therapy in short IUGR children. Solid lines represent data obtained from prepubertal children and dashed lines represent data obtained from pubertal subjects. S_{I} means insulin sensitivity.

FIG. 2 depicts a graph showing changes in the acute insulin response prior to, during and following GH therapy in short children. Solid lines represent data obtained from prepubertal children and dashed lines represent data obtained from pubertal subjects.

FIG. 3 depicts a graph of effects of GH and troglitazone/metformin on fasting plasma insulin levels in IUGR rats. *p<0.0001 and ♦p=0.01

FIG. 4 depicts a graph of effects of GH and troglitazone/metformin on tail systolic BP in IUGR rats. *and ♦p<0.0001

FIG. 5 depicts a graph of effects of GH and troglitazone/metformin on fasting plasma IGF-I levels in IUGR rats. *p<0.0001. Effect of troglitazone/metformin across all groups (∇ and ♦p<0.0001).

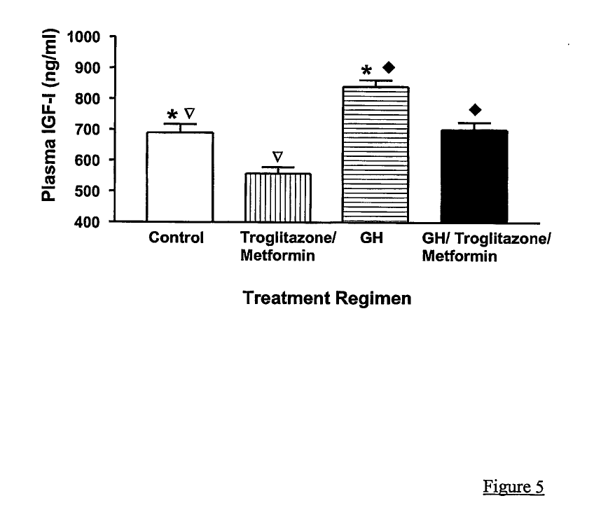

FIG. 6 depicts a graph of effects of GH and troglitazone/metformin on fasting plasma FFA leve IUGR rats. Effect of troglitazone/metformin across all groups (*and ♦p<0.0001).

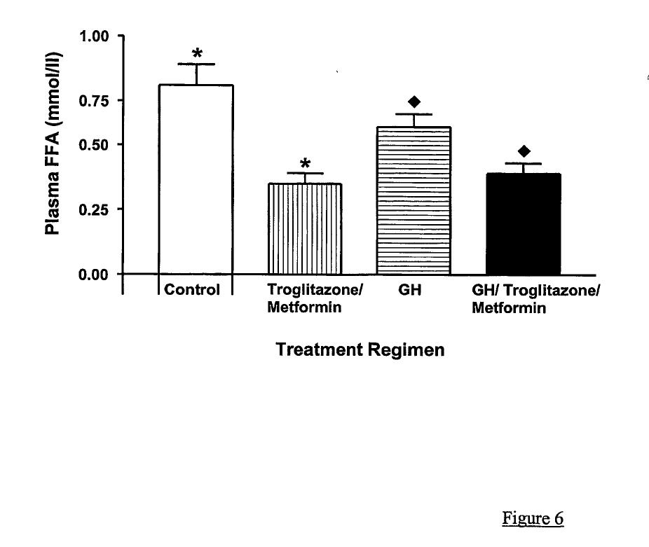

### DETAILED DESCRIPTION THE INVENTION

Embodiments of this invention include methods for the treatment or prophylaxis of adverse consequences of growth hormone (GH) treatment administered to juveniles suffering from growth disorders. GH is commonly used to treat conditions resulting in short stature including but not restricted to growth hormone insufficiency, growth hormone deficiency, Intrauterine Growth Retardation/Restriction, Silver-Russell Syndrome, skeletal abnormalities, chromosomal variations (Turner's syndrome, Down syndrome), or chronic kidney disease related growth retardation. GH treatment has been shown to contribute to a number of conditions such as type 2 diabetes and hypertension. Such conditions have also been observed to be prolonged beyond immediate GH treatment. Such consequences can at least be mitigated, if not completely prevented, by administration of an insulin sensitiser, preferably in combination with the GH treatment. The addition of insulin sensitisers to GH therapy can correct insulin sensitivity to either the pretreatment state or to that of normal children. Where the adverse consequences of growth hormone treatment have not been observed as symptoms, the incidence of the consequences can at least be mitigated prophylactically. Of particular advantage is that while the adverse conditions are at least mitigated, the growth-inducing effect of the GH is not affected by the use of the insulin sensitiser. As a result, the combination treatment provides a useful method of treating the short stature condition (by GH) while at the same time at least reducing some of the adverse consequences of the treatment.

Embodiments of this invention thus include methods of treating short stature conditions and/or methods of prophylaxis or treatment of the adverse effects of the GH treatment. It is particularly relevant to treatment of growth disorders characterised by preexisting insulin resistance. From another perspective, the invention is directed to compositions for use in such methods and to the manufacture of medicaments for such treatments.

The applicants have surprisingly observed that during rhGH treatment of SGA/IUGR children there is a further reduction in insulin sensitivity, which persists three months after rhGH is stopped. The reduction in insulin sensitivity is accompanied by a compensatory increase in insulin secretion, as reflected in the acute insulin response, to maintain euglycemia.

Definitions

As used herein, the term ‘growth hormone’ or ‘GH’, includes growth hormone; growth hormone secretagogues (GHSs); growth hormone releasing proteins/peptides (GHRP); growth hormone releasing hormone (GHRH); somatotropin release inhibitory factor (SRIF); compounds which increase the endogenous release of growth hormone or growth hormone secretagogues; a pharmaceutically acceptable salt of a GHS; analogues; mimetics; functionally equivalent ligands; prodrugs; metabolites; derivatives; agonists; compounds which increase the activity of neural growth hormone receptors; compounds which bind to or increase the concentration of compounds which bind to neural growth hormone receptors; compounds which lessen or prevent inhibition of GH, GHS or ligand activity; or inhibitors of antagonists thereof.

Examples of agents which stimulate growth hormone and production or lessen or prevent its inhibition include but are not limited to growth hormone releasing peptides such as GHRP-1, GHRP-2 (also known as KP-102), GHRP-6, hexarelin, G-7039, G-7502, L-692,429, L-629,585, L-163,191 (aka MK-0677), ipamorelin, NN703, GHS-25, CP-424,391, ghrelin, SM-130686 or GHRH or inhibitors of GH antagonists (substances which bind growth hormone or otherwise prevent or reduce the action of GH within the body). These latter compounds exert an indirect effect on effective GH concentrations through the removal of an inhibitory mechanism and include substances such as somatostatin release inhibitory factor (SRIF).

The GH can be any GH in native-sequence or in variant form and from any source, whether natural, synthetic or recombinant. Examples being human GH, bovine GH, rat GH and porcine GH. It is, however, preferred that the GH employed be human GH.

As used herein, the term ‘growth disorder’ refers to any condition resulting in short stature. Such conditions include but are not limited to growth hormone insufficiency, growth hormone deficiency, Intrauterine Growth Retardation/Restriction, prematurity, skeletal abnormalities, chromosomal variations (Turner's Syndrome, Down Syndrome), or chronic kidney disease related growth retardation, or any other condition resulting in short stature.

As used herein, the term ‘pre-existing insulin resistance’ refers to a growth disorder related reduction in insulin sensitivity existing prior to commencement of GH therapy.

As used herein, the term ‘insulin resistance’ refers to any condition or state whereby the actions of insulin are impaired or reduced.

As used herein, the term ‘hyperinsulinemia’ refers to the production of excessive amounts of insulin.

As used herein, the term ‘insulin sensitiser’ includes biguanides and thiazolidinediones. The biguanides of interest include but are not limited to metformin and buformin. The thiazolidinediones of interest include, but are not limited to, troglitazone, ciglitazone, rosiglitazone and pioglitazone.

Methods of Treatment

In certain embodiments, this invention includes methods of preventing and/or treating the adverse consequences of growth hormone treatment of a growth disorder in a juvenile suffering from a growth disorder, comprising administering an effective amount of growth hormone in combination with an insulin sensitiser or a combination of insulin sensitisers.

In additional embodiments, this invention includes methods of treating growth disorders in a juvenile, comprising administering an effective amount of an insulin sensitiser or a combination of insulin sensitisers in combination with the growth hormone treatment.

Growth hormone is known to antagonise the actions of insulin through multiple steps in the insulin-signalling cascade (Smith et al. 1997). In both acute (e.g. rhGH infusion) and chronic elevations of GH (e.g. acromegaly), insulin sensitivity progressively falls with increasing rhGH levels (Sonksen et al. 1993; Bratusch-Marrain et al. 1982). The mechanism of this rhGH-induced reduction in insulin sensitivity involves multiple sites in insulin receptor signal transduction. Insulin receptor β subunit autophosphorylation, insulin receptor substrate-1 phosphorylation and basal GLUT-4 abundance are all reduced with in vitro human skeletal muscle rhGH exposure. In addition, rhGH reduces in vivo glucose effectiveness. Short-term growth hormone administration in humans and animals induces insulin resistance and glucose intolerance; it has been shown to impair insulin-mediated suppression of hepatic glucose output and increased peripheral glucose utilization (Sugimoto et al. 1998).

The inventors' observations indicate that GH therapy may cause a long term or an irreversible reduction in insulin sensitivity in SGA children who already have a pre-existing impairment of insulin sensitivity. An isolated reduction in insulin sensitivity is a risk factor for the later development of type 2 diabetes mellitus in populations with a high incidence of the disease.

Methods of attenuating insulin resistance are known in prior art. Biguanides (e.g. metformin or phenformin) and thiazolidinediones (or glitazones, e.g. troglitazone, rosiglitazone pioglitazone) are insulin sensitisers used commonly in treatment of NIDDM (non-insulin dependent diabetes mellitus) and insulin resistance. Glitazones are derivatives of thiazolidinedione, and, according to one theory, can act via activation of the peroxisome proliferator-activated receptor (PPAR) γsubtype.

Prior art has suggested the usefulness of thiazolidinediones in attenuating the GH treatment related insulin resistance in healthy rats. The use of troglitazone prior to and together with the recombinant human GH therapy has been shown to counteract the insulin-antagonistic action of rhGH in the liver and the peripheral tissues (Sugimoto et al. 1999; Sugimoto et al. 1998). In another study, pioglitazone, an analog of thiazolidinedione, was administered short-term prior to and together with recombinant human GH to insulin-resistant 3-6 month old obese (ob/ob homozygous genotype) mice (Towns R et al. 1994). The study was designed to establish whether pioglitazone could inhibit and ameliorate the negative, insulin desensitizing, effects of GH therapy. Pioglitazone was shown to block the ability of hGH to increase blood glucose and plasma insulin levels in mouse model of obesity. The authors of the study showed that administration of pioglitazone did not affect the GH induced weight gain of the adult ob/ob mice.

The novel idea comprising administration of an insulin sensitiser in combination with GH therapy to treat growth disorders in juveniles was not previously described in the prior art. The studies known in the prior art were carried out in metabolic conditions different to those present in SGA juveniles and thus the published findings are not predictive of the applicability, utility and efficacy of the combination treatment in children with growth disorders. Prior art studies investigated short-term consequences of insulin sensitizers and GH co-treatment (the compounds co-administered for periods no longer than 3 days). The novel idea comprising long-term administration of an insulin sensitiser in combination with GH therapy to treat growth disorders in juveniles is not rendered obvious by the known short-term use of GH and insulin sensitisers in models of obesity or healthy animal models.

Some embodiments of the present invention are based on in the surprising finding that long term GH and insulin sensitizer co-treatment administered to SGA juveniles ameliorates the adverse effects of the GH mono-therapy. Significantly, the desired GH-induced increase in linear growth in SGA juveniles is not decreased by the administration of the co-therapy. This novel method of treatment of growth disorders can increase insulin sensitivity, prevent fasting hyperinsulinemia, ameliorate pre-existing growth disorder related insulin resistance and insulin resistance associated with GH therapy. In addition, treatment with GH and insulin sensitisers can lead to a marked improvement in metabolic indicators, including reduction in fasting insulin levels, reduction in systolic blood pressure, reduction in FFA and reduction in retroperitoneal fat.

The novel application provides new methods and compositions aimed at alleviating the conditions associated with GH therapy in juveniles and enhancing the efficacy of the methods existing in the prior art. Moreover, the novel methods disclosed herein provide the public with beneficial alternatives to the methods existing in the prior art.

Useful insulin sensitisers include biguanides and/or thiazolidinediones. Of particular interest is the biguanide metformin and/or the thiazolidinediones troglitazone, ciglitazone, pioglitazone and rosiglitazone or a combination of a biguanide and a thiazolidinedione. Combined metformin and troglitazone therapy, when compared to either therapy alone, can reduce fasting and post-prandial glucose concentrations in adults with type 2 diabetes mellitus by an additional 20% (Inzucchi et al. 1998). These two insulin sensitisers may have complimentary modes of action that allow for this additive effect on glucose levels to occur. According to one theory, metformin may act by decreasing endogenous hepatic glucose production and troglitazone may act by increasing the rate of peripheral glucose disposal (Izucchi et al. 1998). Thiazolidinediones are synthetic ligands for peroxisome proliferator-activated receptor gamma (PPARγ) (Forman et al. 1995). Troglitazone has been shown to counteract the insulin-antagonistic action of rhGH in liver and peripheral tissues (Sugimoto et al. 1999). Interestingly, GH therapy has been shown to impair insulin-mediated suppression of hepatic glucose output and increased peripheral glucose utilization (Sugimoto et al. 1998). Thus, combination therapy with a biguanide and a thiazolidinedione can prevent or diminish two major GH-induced adverse effects on glucose homeostasis.

In additional embodiments, the present invention includes methods of preventing, delaying or treating diabetes mellitus, dyslipidemia, hypertension and/or obesity in mammals suffering from growth disorders. Troglitazone and metformin can independently lower blood pressure of patients with hypertension and insulin resistance following improvement in insulin sensitivity (Nolan et al. 1994, Carlsen et al. 1996). Troglitazone can also induce vasodilation. Although not all mechanisms of this action are known, one mechanism is through the prostaglandin pathway (Walker et al. 1998). We found that troglitazone and metformin can lower systolic BP with or without GH therapy, which can be of importance in the management of insulin resistant IUGR patients with hypertension. In diabetic patients, troglitazone can exhibit a differential effect on adipose tissue compartments with a reduction in visceral fat and an increase in subcutaneous fat (Kawai et al. 1999). GH and insulin sensitisers were associated with an additive effect in the reduction in retroperitoneal fat. In addition, the animals that received insulin sensitisers were leaner. Therefore, it is likely that GH treated children with growth disorders who also receive a thiazolidinedione benefit from reduction in visceral fat, which is associated with increased insulin sensitivity.

In some embodiments, an insulin sensitizer is selected from group of biguanides and thiazolidinediones, and in some cases, a combination of a biguanide and a thiazolidinedione can be useful when administered to a juvenile, for example, during the growth hormone treatment

In additional embodiments, the present invention provides for the use of growth hormone and an insulin sensitiser or a combination of insulin sensitisers in the preparation of a medicament or composition for treating growth disorders and/or preventing or treating the adverse consequences of growth hormone treatment.

Pharmaceutical Composition

In general, compounds of this invention will be administered as pharmaceutical compositions by one of the following routes: oral, topical, systemic (e.g. transdermal, intranasal, or by suppository), parenteral (e.g. intramuscular, subcutaneous or intravenous injection), by implantation and by infusion through such devices as osmotic pumps, transdermal patches and the like. In some embodiments, the GH can be administered daily through a subcutaneous injection In other embodiments, the insulin sensitizer or a combination of insulin sensitizers can be administered orally.

Compositions may take the form of tablets, pills, capsules, semisolids, powders, sustained release formulations, solutions, suspensions, elixirs, aerosols or any other appropriate compositions; and may include pharmaceutically acceptable excipients. Suitable excipients are well known to persons of ordinary skill in the art, and they, and the methods of formulating the compositions, may be found in such standard references as Gennaro A R: Remington: The Science and Practice of Pharmacy, 20^{th} Ed., Lippincott, Williams and Wilkins, Philadephia, Pa. (2000). Suitable liquid carriers, especially for injectable solutions include water, aqueous saline solutions, aqueous dextrose solutions and the like, with isotonic solutions being desirable for parenteral administration.

Compounds of this invention are also suitably administered by a sustained-release system. Suitable examples of sustained release compositions include semi-permeable polymer matrices in the form of shaped articles e.g films or microcapsules. Sustained release matrices include polylactides (U.S. Pat. No. 3,773,919; EP 58,481), copolymers of L-glutamic acid and gamma-ethyl-L-glutamate, poly(2-hydroxyethyl methacrylate), ethylene vinyl acetate or poly-D-(−)-3-hydroxybutyric acid (EP 133,988). Sustained-release compositions also include a liposomally entrapped compound. Liposomes containing the compound are prepared by methods known per se: DE 3,218,121; EP 52,322; EP 36,676; EP 88,046; EP 143,949; EP 142,641; Japanese Pat. Apln. 83-118008; U.S. Pat. Nos. 4,485,045 and 4,544,545 and EP 102,324. Compounds of this invention may also be PEGylated to increase their lifetime in vivo, based on e.g. the conjugate technology described in WO 95/32003. However, it can be readily appreciated that other sustained-release preparations can be advantageously used with the compositions and methods of this invention.

### EXAMPLES

The following examples are provided for illustration only and are not intended to limit the scope of this invention. Other embodiments and variations are possible based on the discoveries disclosed herein, and all such embodiments and variations are considered to be within the scope of this invention.

### EXAMPLE 1

### Effects of Growth Hormone and Insulin Sensitisers in Children Small for Gestational Age

Objectives

A purpose of this study was to determine the effects of human GH therapy on insulin sensitivity in short children born small for gestational age (SGA). To carry out these studies, we used recombinant human growth hormone (rhGH).

Subjects

All subjects were born SGA, had marked short stature (height approximately—3 standard deviation score (SDS)) with a height velocity<25th percentile for >1 year prior to starting rhGH therapy. SGA was defined as a birth weight<10th percentile for gestational age. Additional enrolment criteria for inclusion into the study included; no change in Tanner pubertal stage and/or testicular volumes throughout the study period, normal GH response to clonidine stimulation (defined as a GH level≧7 μgm/L), absence of both islet cell antibodies (<10 Juvenile Diabetes Foundation units) and insulin autoantibodies to exclude type 1 prediabetes. Prepubertal subjects were defined as males with a testicular volume<4 ml and girls with tanner stage 1 breast development. Subjects were excluded if; a chromosomal, intrauterine infection or syndromal cause for SGA was identified, a first degree relative had type 2 diabetes mellitus, or medical therapy was taken that was known to influence SI. Subjects were recruited from the Endocrinology Clinics at Starship Children's Hospital. Birth weight and length were converted into SDSs to correct for age and sex. Body mass index (BMI) was used as a measure of relative obesity and expressed as BMI SDS.

Methods

Insulin sensitivity (S_{I}) was measured in 11 subjects prior to and 12 subjects during rhGH treatment. In addition five prepubertal subjects agreed to have rhGH therapy suspended for three months with reassessment of S_{I}. Bergman's minimal model was used to measure S_{I}, acute insulin response (AIR) and glucose effectiveness (Sg). The data was provided from a 90 min frequently sampled iv glucose tolerance test with tolbutamide, which had been modified and validated for use in children as previously described (Cutfield et al. 1990). S_{I} is a measure of insulin mediated glucose uptake, AIR a measure of insulin secretory capacity and Sg is a measure of glucose stimulated glucose uptake independent of insulin. These three indices are characteristic measurements useful for determining glycemic states. Plasma glucose and insulin was measured from all samples and the values used for measurement of S_{I}, AIR and Sg. Approval for the study was provided by the North Health Ethics Committee and signed, informed consent was obtained from subjects and their parents.

Assays

Plasma glucose concentration was measured using a Hitachi 911 automated random access analyser (Tokyo, Japan) with an interassay coefficient variation of 1.2% (Trinder P. Determination of blood glucose using an oxidase-peroxidase system with a non-carcinogenic chromogen. J Clin Pathol 1995; 22: 158-61). Insulin was determined by Abbotts IMX microparticle enzyme immunoassay with an interassay coefficient of variation of <5%. Plasma IGF-I was measured using an established IGF binding protein blocked radioimmunoassay (Blum et al. Radioimmunoassays for insulin-like growth factors and their binding proteins. Growth Reg. 1994; 4: (Suppl.) 11-19). The IGF-I detection limit was 1.3 ng/ml with intra-assay and inter-assay coefficients of variation of 5.0% and 9.2%, respectively. Plasma IGF-II samples were intially treated with acid-ethanol cryoprecipitation to remove IGF II from binding proteins. IGF II was then measured by RIA using a highly specific polyclonal antiserum (Blum et al. A specific radioimmunoassay for insulin-like growth factor II. The interference of IGF binding proteins can be blocked by excess IGF-I. Acta Endcrinol (Copenh) 1988; 129:427-435). Residual IGF binding proteins were blocked by an excess of IGF-I. Inter- and intra-assay coefficients of variation were 9.7% and 5.1%, respectively, and cross-reactivity with IGF-1 and insulin were <0.005%. A validated double-antibody RIA was used for measurement of leptin in human plasma (Vickers et al. Fetal origins of hyperphagia and hypertension and postnatal amplification by hypercaloric nutrition. Am J Physiol (Endocrinol Metab) 2000; 279; E83-87). Human leptin (No. CR09780 Crystal Chem, Houston, Tex.) standards were used in concentrations ranging from 0.5 to 50 ng/ml. The intra-assay coefficient of variation and the inter-assay coefficient of variation were 5% and 11%, respectively. Insulin autoantibodies were measured by a competitive radioimmunoassay (Vardi et al. Prospective evaluation of subjects at high risk for development of type 1 diabetes mellitus. Diabetes 1987; 36: 1286-91) and islet cell antibodies by indirect immunofluorescence (Pilcher et al. A sensitive and reproducible method for the assay of human islet cell antibodies. J Immunol Methods 1990; 129: 111-7).

Analysis

Area under the curve (“AMR”) was measured as the sum of trapeziums corrected for baseline using the formula: AIR=0.5×0 min+1.5×2 min+3 min+4 min+5 min+1.5×6 min+2×8 min+1.5×10 min−10×0 min.

Analysis was carried out using the statistical package SAS (version 8) for personal computers. Paired t-tests were used to compare variables prior to and during GH therapy. Pearsons correlations were used to investigate the relationships between changes in S_{I} and other variables affected by rhGH therapy. A p value of <0.05 was defined as significant. Values are expressed as means with SEM or as means of the differences when comparing changes in variables.

Results

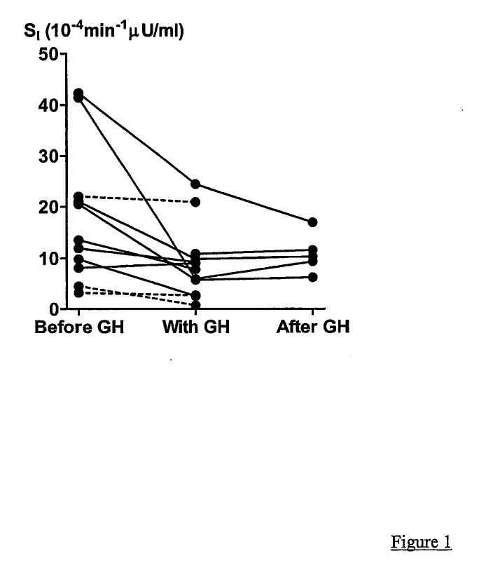

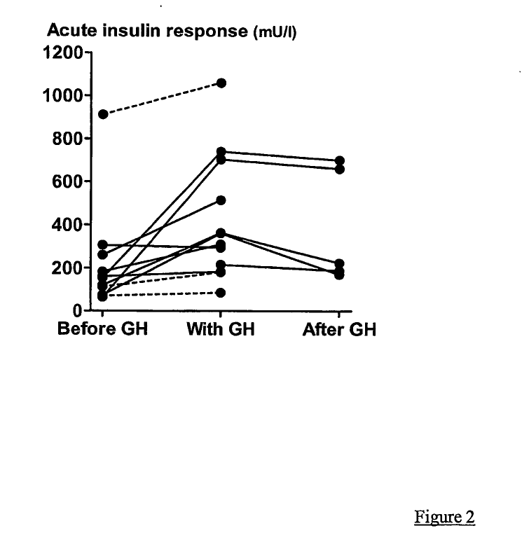

Seven males and five females were studied. Three subjects were in early puberty (Tanner stage 2 breast development or testicular volumes<6 ml) on both occasions when insulin sensitivity was measured. All other subjects remained prepubertal, as earlier defined, throughout the study period. At enrollment subjects were 9.3±1.0 yrs old, of Caucasian ethnicity (11 of 12 subjects), with a birth weight SDS of −3.5±0.7 and pretreatment height SDS of −3.2±0.1. Subjects received 21±6 months of rhGH treatment prior to reassessment of S_{I}. Body mass index SDS did not change with rhGH therapy (−0.7±0.5 before versus −0.7±0.5 during rhGH treatment). During rhGH therapy S_{I} values fell 44±10% (p=0.018) (FIG. 1) with an insignificant difference in prepubertal children compared to pubertal subjects (48% versus 35%). There was a compensatory rise in the AIR of 123±59% (p<0.009) (FIG. 2). There was no change in Sg with rhGH therapy with values of 2.2±0.9 10⁻²min⁻¹ prior to rhGH therapy and 2.2±0.6 10⁻²min⁻during treatment. There was no recovery in S_{I} in the five prepubertal subjects studied three months after rhGH therapy was suspended (FIG. 1).

One subject (subject 1) developed type 2 diabetes mellitus at the age of 18.0 years, 12 months after cessation of 4.1 years of rhGH therapy. Prior to rhGH therapy his BMI SDS was 1.8 and at diagnosis of diabetes mellitus his BMI SDS had increased to 3.2, with the presence of acanthosis nigricans. He did not have a family history of type 2 diabetes mellitus, nor did he have detectable islet cell antibodies nor insulin autoantibodies.

Overall, rhGH therapy was associated with a rise in fasting serum IGF-I levels from 127±37 μgm/l to 189±46 μgm/l, p<0.001. However, there was no change in fasting serum leptin (from 10.9±5.6 pretreatment to 7.1±3.0 mg/l with treatment) nor IGF-II levels (from 539±52 pretreatment to 566±35 μgm/l with treatment) with rhGH therapy.

There was no correlation between the degree of fall in insulin sensitivity and acceleration in growth (as represented by the change in height velocity SDS over the first year of therapy (11±0.6 mos)), with rhGH therapy (r²=0.16, p=0.2). There was also no correlation between the fall in insulin sensitivity and the increase in IGF-I level with rhGH therapy (r²=0.09, p=0.4).

Conclusions

Growth hormone therapy of short SGA children leads to an isolated reduction in insulin sensitivity that does not spontaneously reverse, even after three months after the cessation of rhGH therapy.

### EXAMPLE 2

### Effects of Insulin Sensitizers on GH-Induced Insulin Resistance in Rats

In another series of experiments, we determined the effects of insulin sensitisers on insulin resistance induced by GH in rats in vivo. These experiments were approved by the Animal Ethics Committee of the University of Auckland.

Animal Model

Studies of insulin resistance in vivo in rats were carried out in animals in which insulin resistance was produced by fetal undernutrition (“IUGR”) as described previously (Woodall et al. 1996). Virgin Wistar rats (age 100±5 days) were time mated using a rat estrus cycle monitor to assess the stage of estrus of the animals prior to introducing the male. After confirmation of mating, rats were housed individually in standard rat cages containing wood shavings as bedding and free access to water. All rats were kept in the same room with a constant temperature maintained at 25° C. and a 12-h light: 12-h darkness cycle. Animals received an undernutrition diet throughout pregnancy that was 30% of an ad libitum (“AD”) diet. Food intake and maternal weights were recorded daily until birth. A total of 40 male pups were recruited for the study. After birth, pups were weighed and litter size recorded. Litter size was then adjusted to eight pups to ensure standardised nutrition. Male pups from undernourished mothers were cross-fostered onto dams which received AD feeding throughout pregnancy to ensure post-natal viability. After weaning all male offspring were fed a hypercaloric diet ad libitum for the remainder of the study (total digestible energy 4846 kcal/kg, protein 31.8%, fat 30%, fat/energy ratio 55.72%, protein/energy ratio 26.25%). The fat content of the hypercaloric diet was chosen to be typical of many Western diets known to be associated with abnormalities in glucose metabolism in humans.

Study Protocol

At 25 days of age, three days after weaning, study animals were enrolled into treatment groups for 28 days. The study groups were weight matched. The 40 study males were divided into four treatment groups, each of 10 animals; (a) no treatment (control group), (b) troglitazone and metformin, (c) recombinant bovine GH (GH) and (d) GH, troglitazone and metformin. To evaluate systolic BP in untreated IUGR rats, systolic BP was compared to values in 10 normal male rats at study completion. The mothers of the normal rats were fed ad libitum diets throughout pregnancy.

Troglitazone was added to food at approximately 100 mg/kg/day based on daily food consumption. Metformin was mixed with drinking water and the dose calculated to 500 mg/kg/day based on daily water consumption. Bovine GH was administered subcutaneously at a dose of 10 μgm/gm/day in two divided doses at 0800 and 1700 hours. Body weight and food intake of all offspring were measured daily for the first 2 weeks then every second day until study completion. All drug doses were recalculated every 2 days during the study to ensure accurate dosing during the treatment period.

At day 53, systolic blood pressure measurements were recorded using tail cuff plethysmography (Blood pressure analyser IITC, Life Science, Woodland Hills, Calif., USA) as described previously (Vickers et al. 2000). Rats were restrained in a clear plastic tube in a warmed room (25-28° C.). After the rats had acclimatised (for 10-15 min), the cuff was placed on the tail and inflated to a pressure of 240 mmHg. Pulses in pressure were recorded during deflation at a rate of 3 mmHg/sec and the reappearance of a pulse was used to determine systolic blood pressure. A minimum of three systolic blood pressure recordings were taken per animal. The coefficient of variation for repeated measurements was <5%. The animals were then fasted overnight and sacrificed by halothane anaesthesia followed by decapitation. Blood was collected into heparinised vacutainers and stored on ice until centrifugation and removal of plasma for analysis.

Assays

Assays were carried on samples obtained from fasting animals.

IGF-I in rat blood plasma was measured using an IGF binding protein (IGFBP)-blocked rat RIA described previously (Blum et al. 1994). The half-maximal binding was observed at 0.1 ng/tube and the intra- and inter-assay coefficients of variation were 4.6% and 8.7%, respectively. Rat insulin was measured by an in-house radioimmunoassay (Vickers et al. 2000, Lewis et al. 1999). The half-maximal binding was observed at 0.5 ng/ml and intra-assay coefficient of variation <5%. An in-house radioimmunoassay was used for measurement of leptin in rat plasma (Vickers et al. 2000). The half-maximal binding was observed at 0.37 ng/ml and the intra-assay coefficient of variation was <5% (all samples measured within a single assay).

Plasma glucose and lactate concentrations were measured using a YSI Glucose Analyzer (Model 2300, Yellow Springs Instrument Co., Yellow Springs, Ohio, US). All other blood measurements were performed on a BM/Hitachi 737 analyser by Auckland Healthcare Laboratory Services.

Statistical comparisons were carried out using the statistical package SAS (version 8) for personal computers. Analysis of variance was used to investigate the difference in levels between the four treatments with, specific contrasts used to test the hypotheses of interest. A log transformation was used for analysis of insulin and free fatty acid data. For analysis of growth and body composition data (BMI and % retroperitoneal fat), each of the four treatment groups were analysed as separate treatments and then Dunnetts test was used to compare the three active treatments with the control group. Data are expressed as mean±SEM, with p<0.05.

Results

When the IUGR animals were compared to 10 male offspring of mothers fed ad libitum throughout pregnancy, birth weights (4.0±0.03 versus 6.1±0.04 gm respectively, p<0.0001) and birth lengths (nose to anus lengths 39.9±0.4 mm versus 46.2±0.3 cm respectively, p<0.0001) were considerably lower in the IUGR animals. There was no difference in food intake between the four treatment groups throughout the study period.

Serum insulin levels were substantially and significantly increased in animals that received GH therapy alone (p<0.0001) as shown in FIG. 1. However, insulin sensitisers reduced insulin levels in animals treated with GH compared to animals receiving GH alone (p=0.01). In contrast, insulin sensitisers had little effect in animals not receiving GH. GH treatment was associated with increased plasma glucose, with levels of 11.9±0.9 mmol/l in the GH treated group compared to 7.8±0.3 mmol/l in the no treatment group (p<0.0001).

Systolic BP was higher in the IUGR no treatment group when compared to male offspring from ad libitum fed mothers the normal birth weight no treatment group (120±1.8 versus 114±1.8 mm Hg, p<0.05). GH therapy led to a trend of increasing systolic BP (p=0.06) as shown in FIG. 2. However insulin sensitisers led to a marked reduction in systolic BP when compared to the no treatment group and the GH treated group (p<0.0001).

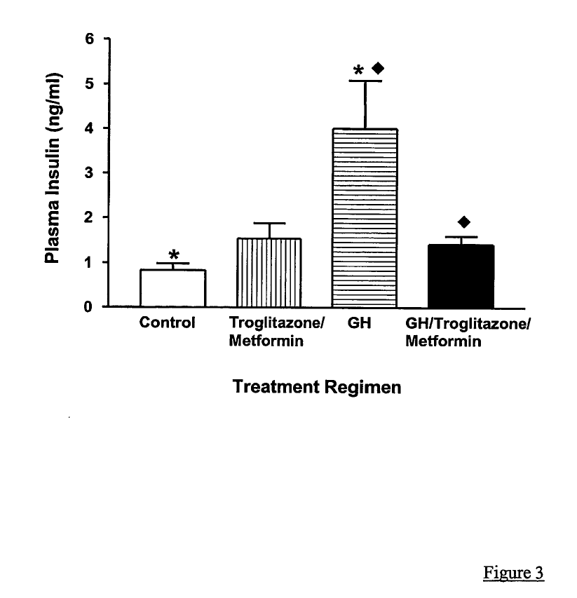

Serum IGF-I levels were higher in the animals who received GH treatment as shown in FIG. 3 (p<0.0001). Surprisingly, IGF-I levels were lower in the animals that received insulin sensitisers and GH compared to those that received GH without insulin sensitisers (p<0.0001). The GH treated groups exhibited a greater growth rate during the study period when compared to those not treated with GH (p<0.008), as illustrated by the GH treatment alone group (nose to anus length 188±1 mm) compared to the no treatment group (179±2 mm). The addition of insulin sensitiser treatment to GH therapy did not influence the growth rate when compared to GH therapy alone (184±3 mm versus 188±1 mm).

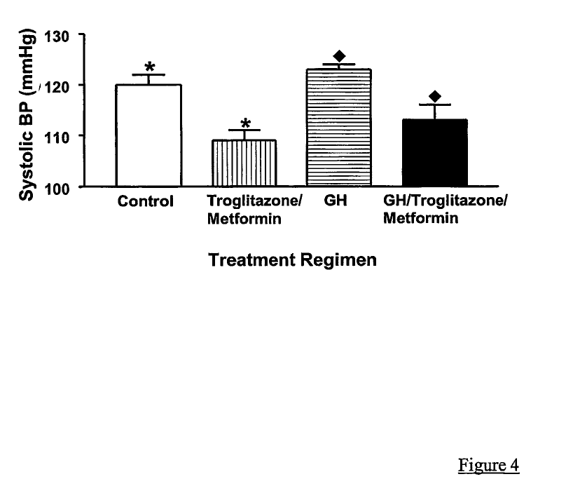

Insulin sensitiser therapy was associated with decreased plasma free fatty acids (FFA) (p<0.0001), most striking in the group that did not receive GH therapy (FIG. 4). The addition of insulin sensitisers to GH therapy caused a greater decrease in FFA than GH therapy alone (p=0.03), however there was no evidence that this was different from the decrease with insulin sensitisers alone (p=0.5).

Body composition was examined in several different ways; body mass index (BMI), retroperitoneal fat expressed as a percentage of body weight (retroperitoneal fat %) and gonadal fat expressed as a percentage of body weight (gonadal fat %). BMI is an established method of assessment of relative adiposity in rats (Maffei et al. 1995, Wade et al. 2000, van den Brandt et al. 2000) and is predictive of similar effects in humans. Insulin sensitisers were associated with a reduction in BMI, retroperitoneal fat % but not gonadal fat %. Although GH therapy was not associated with a change in BMI, insulin sensitiser treatment was associated with a reduction in BMI across all groups (p=0.009). BMI values were 6.1+0.1 versus 6.8±0.2 for the insulin sensitiser versus no treatment groups, respectively, and 6.3±0.2 versus 6.9±0.1 for the GH and insulin sensitisers versus the GH treatment groups, respectively. GH therapy was associated with a reduction in retroperitoneal fat % (p=0.0007). However, insulin sensitisers were associated with a greater reduction in retroperitoneal fat %. The insulin sensitiser group had lower retroperitoneal fat % than the no treatment group (0.37±0.04% versus 0.97±0.06%, respectively, p<0.0001) and the GH and insulin sensitiser-treated group had lower retroperitoneal fat % than the GH treatment group (0.28±0.06% versus 0.69±0.05%, respectively, p<0.0001). While GH treatment was associated with an increase in gonadal fat % across the four groups (p=0.035), insulin sensitiser treatment was not associated with changes in gonadal fat % (p=0.6).

Discussion

Treatment with insulin sensitisers in juvenile IUGR rats decreased or prevented fasting hyperinsulinemia, an important adverse effect of GH therapy. In addition, insulin sensitisers lowered the elevated blood pressure of control, untreated IUGR rats. Although the mechanisms responsible for these effects are uncertain, according to one hypothesis, in non-diabetic, euglycemic humans and fasting animals, hyperinsulinemia reflects a generalised increase in insulin secretion that is a compensatory response to a reduction in insulin sensitivity (Kahn et al. 1993, Bergman 1989). Therefore, the hyperinsulinemia we observed in GH treated rats can be an indicator of reduced insulin sensitivity.

SGA children with IUGR have a preexisting reduced insulin sensitivity that is further reduced with recombinant GH therapy (Cutfield et al. 2001). There was no recovery of insulin sensitivity three months after recombinant GH therapy was discontinued. Our study indicates that the addition of insulin sensitisers to recombinant GH therapy prevented the GH-induced insulin resistance. Because in vivo studies in rats are predictive of human responses, we conclude that combined therapy with GH and insulin sensitisers in SGA children will decrease or prevent the development of insulin resistance induced by recombinant GH therapy without diminishing the desirable growth-stimulating effects of GH.

The fall in IGF-I levels with insulin sensitiser therapy with or without GH therapy was an unexpected observation. The hyperinsulinemia seen in IUGR children correlated with elevated IGF-I levels. Although the mechanisms underlying the relationship are not known with certainty, according to one theory, the reduction in fasting insulin levels in IUGR rats treated with insulin sensitizers may have decreased IGF-I values. Consistent with this theory, IGF-I levels have been shown to be associated with nutritional status with thin children exhibiting reduced IGF-I levels (Soliman et al. 1986, Juul et al. 1995). The rats treated with insulin sensitisers were leaner, which could also explain the lower IGF-I values. Importantly, the lower IGF-I values observed with insulin sensitisers were not due to hepatic dysfunction. Thus, measurement of IGF-I levels can be predictive of the efficacy of treatment using combined GH and insulin sensitisers.

Our observation that insulin sensitiser therapy was associated with a marked reduction in free fatty acid (FFA) levels is completely unexpected based on the report of Sugimoto et al. who found with the same dose of troglitazone a small, insignificant fall in FFA with associated with troglitazone and GH therapy to normal rats (Sugimoto et al. 1998). Similarly, a fall in FFA in normal animals would be expected given that thiazolidinediones are synthetic ligands for peroxisome proliferator-activated receptor gamma (PPARγ) (Forman et al. 1995).

In the fed state, activation of PPARγ in rats leads to greater FFA uptake into adipocytes. It has been proposed that increasing the uptake of FFA into adipocytes diverts FFA away from skeletal muscle (Kersten et al. 2000). FFA can impair insulin mediated glucose uptake in skeletal muscle, thus an increase in circulating FFA is associated with a reduction in insulin sensitivity (Felber et al. 1964, Reaven et al. 1988, Randle et al. 1963). Although not known with certainty, one theory for the actions of thiazolidinediones, as described herein, to improve insulin sensitivity is via greater uptake of FFA. The fall observed in plasma FFA with GH therapy was an unexpected observation given that some of the insulin antagonistic effects of GH are thought to be due to increased lipolysis and subsequent elevation in plasma FFA leading to inhibition of glucose uptake (Moller et al. 1987).

The elevated systolic BP that we observed in the untreated IUGR rats is consistent with other IUGR rat studies and according to one theory, may be due to insulin resistance and hyperinsulinism (Vickers et al. 2000, Woodhall et al. 1996). Insulin resistance and secondary hyperinsulinism are important in the pathogenesis of hypertension, which occurs more commonly in adults of low birth weight (Barker et al. 1993, Law et al. 1991). Insulin has an important vasodilatory function that is mediated through nitric oxide release (McNally et al. 1995, Steinberg et al. 1994). Insulin-induced vasodilation is impaired in disorders characterised by insulin resistance (Laakso et al. 1992, Laakso et al. 1993, Feldman et al. 1993). Both troglitazone and metformin have been shown to independently lower blood pressure of patients with hypertension and insulin resistance following improvement in insulin sensitivity (Nolan et al. 1994, Carlsen et al. 1996). Troglitazone has also been shown to induce vasodilation by an additional mechanism through the prostaglandin pathway (Walker et al. 1998).

In addition, the animals that received insulin sensitisers were leaner. Retroperitoneal fat is a major component of abdominal fat in rodents and has been used as a marker of visceral fat (Hida et al. 2000, Rasmussen et al. 1999). In humans, abdominal fat, or more precisely visceral fat, is a major risk factor for the development of glucose intolerance, dyslipidemia, hypertension and atherosclerotic coronary artery disease (Kaplan 1989, Matsuzawa et al. 1993). There is a greater risk of abdominal obesity for men born of low birth weight, which includes those with IUGR (Byberg et al. 2000). In diabetic patients troglitazone has been shown to exhibit a differential effect on adipose tissue compartments with a reduction in visceral and an increase in subcutaneous fat (Kawai et al. 1999). Reduction in visceral fat has been demonstrated to markedly improve insulin sensitivity and reduce hepatic glucose output (Kawai et al. 1999, Cases et al. 2000). Therefore, patients undergoing GH therapy in combination with a thiazolidinedione or other insulin sensitizers can benefit from reduction in visceral fat, which will further improve insulin sensitivity.

Conclusion

Insulin sensitiser therapy is effective in attenuating GH induced hyperinsulinemia in an insulin resistant IUGR model. During GH therapy, insulin sensitiser therapy is associated with an improvement in metabolic parameters that included reduction in fasting insulin, systolic blood pressure, FFA and retroperitoneal fat. The addition of insulin sensitisers to recombinant GH therapy prevents the reduction in insulin sensitivity induced by recombinant GH therapy while GH-induced acceleration in growth is not constrained. Because of the similarity in glucose and fat metabolism of rats and human beings, the in vivo results obtained in rats are predictive of efficacy of treatment of human beings with growth hormone and insulin sensitizers.

The foregoing describes the invention including preferred forms thereof. Alterations and medications that would be apparent to the skilled person are intended to be included within the spirit and scope of the invention disclosed.

Bibliography

Azcona C, Albanese A, Bareille P, Stanhope R. 1998. Growth hormone treatment in growth hormone-sufficient and -insufficient children with intrauterine growth retardation/Russell Silver Syndrome. Hormone Research 50: 22-7.
Barker D J, Hales C N, Fall C H, Osmond C, Phipps K, Clark P M. 1993. Type 2 (non-insulin-dependent) diabetes mellitus, hypertension and hyperlipidemia (Syndrome X): relation to reduced foetal growth. Diabetologia 36: 62-7.
Blum W F, Breier B H. 1994. Radioimmunoassay for IGFs and IGFBPs. Growth Regulation 4: 11-19.
Bratusch-Marrain et al. 1982. The effect of growth hormone on glucose metabolism and insulin secretion in man. J Clin Endocrinol Metab; 55: 973.
Byberg L, McKeigue P M, Zethelius B, Lithell H O. 2000. Birth weight and the insulin resistance syndrome: association of low birth weight with truncal obesity and raised plasminogen activator inhibitor-1 but not with abdominal obesity or plasma lipid disturbances. Diabetologia 43(1):54-60.
Caprio et al. Effect of growth treatment on hyperinsulinemia associated with Turner Syndrome. J Pediatr 1992; 120: 238-43.
Carlsen S M, Rossoll O, Bjerve K S, Folling I. 1996. Metformin improves blood lipid pattern in non-diabetic patients with coronary artery disease. Journal of Internal Medicine 239(3): 227-233.
Cases J A, Barzilai N. 2000. The regulation of body fat distribution and the modulation of insulin action. International Journal of Obesity and Related Metabolic Disorders 24 (Suppl) 4:S63-6.
Cutfield et al. Reduced insulin sensitivity during growth hormone therapy for short children born small for gestational age. Unpublished. 2001.
Cutfield W S, Hofman P L, Jackson W E, Rolfe G, Robinson E M, Breier B H, Vickers M. Reduced insulin sensitivity during GH therapy in IUGR children. Oral presentation at International Congress of Endocrinology 2000. Sydney, Australia November 2000.
Cutfield W S, Wilton P, Bennmarker H, Albertsson-Wikland K, Chatelain P, Ranke M B, Price D A. 2000. The incidence of diabetes mellitus and inpaired glucose tolerance in children and adolescents receiving growth honnone treatment. The Lancet; 355: 610-13.
Cutfield W S, Bergman R N, Menon R K, Sperling M A. The modified minimal model: application to measurement of insulin sensitivity in children. J Clin Endocrinol Metab 1990; 70:1644-50.
De Zegher F, Albertsson-Wikland K, Wollman H A, Chatelain P, Chaussain J L, Lofstrom A et al. 2000. Growth hormone treatment of short children born small for gestational age: growth responses with continuous and discontinuous regimens over six years. Journal of Clinical Endocrinology and Metabolism 85: 2816-21.
De Zegher F, Maes M, Gargosky S E, Heinrichs C, Du Caju M U L, Thiry G et al. 1996. High dose growth hormone treatment of short children born small for gestational age. Journal of Clinical Endocrinology and Metabolism 81: 1887-92.96
Felber J P, Vannotti A. 1964. Effect of fat infusion on glucose tolerance and insulin plasma levels. Medical Experimentation 10: 153-7.
Feldman R D, Briebier G S. 1993. Insulin-mediated vasodilation: impairment with increased blood pressure and body mass. The Lancet 342: 707-9.
Fjellestad-Paulsen A, Czernichow P, Bost M, Colle M, Lebouc J Y, Lecornu M, Leheup B, Lima J M, Raux M C, Toublanc J E, Rappaport R. 1998. Three year data from a comparative study with recombinant growth hormone in the treatment of short stature in young children with intrauterine growth retardation. Acta Paediatrica 87: 511-7.
Forman B M, Tontonoz P, Chen J, Brun R P, Spiegelman B M, Evans R M. 1995. 15-Deoxy-Δ^{12,14}-prostaglandin J₂ is a ligand for the adipocyte determination factor PPARγ. Cell 83: 803-12.
Heptula et al. 1997. Decreased insulin sensitivity and compensatory hyperinsulinemia after hormone treatment in children with short stature. J Clin Endocrinol Metab 82: 3234-8.
Hida K, Wada J, Hirakushi K, Tsuchivama Y, Shikata K, Makino H. 2000. Identification of genes specifically expressed in the accumulated visceral adipose tissue of OLETF rats. Journal of Lipid Research 41(10): 1615-22.
Hofman P L, Cutfield W S, Robinson E M, Bergman R N, Menon R K, Sperling M A, Gluckman P D. 1997. Insulin resistance in short children with intrauterine growth retardation. Journal of Clinical Endocrinology and Metabolism 82: 402-6.
Inzucchi S E, Maggs D G, Spollet G R, Page S l, Rife F S, Walton V, Shulman G I. 1998. Efficacy and metabolic effects of metformin and troglitazone in type 2 diabetes mellitus. New England Journal of Medicine 338: 867-72.
Juul A, Dalgard P, Blum W F, Bang P, Hall K, Michaelsen K F, Muller J, Skakkebaek N E. 1995. Serum levles of insulin-like growth factor binding protein-3 in healthy infants, children and adolescents: the relation to IGF-I, IGF-II, IGFBP-1, IGFBP-2, age, sex, body mass index, and pubertal maturation. Journal of Clinical Endocrinology and Metabolism 80: 2534-42.
Kahn S E, Prigeon R L, McCulloch D K Boyko E J, Bergrnan R N, Schwartz M W, Neifing J L, Ward W K, Beard J C, Palmer J C. 1993. Quantification of the relationship between insulin sensitivity and beta cell function in human subjects. Diabetes 42: 1663-72.
Kaplan N M. 1989. The deadly quartet: upper body obesity, glucose intolerance, hyperglyceridemia and hypertension. Archives of Internal Medicine 149: 1514-20.
Kawai T, Takei I, Oguma Y, Ohashi N, Tokui M, Oguchi S, Katsukawa F, Hirose H, Shimada A, Watanabe K, Saruta T. 1999. Effects of troglitazone on fat distribution in the treatment of male type 2 diabetes. Metabolism 48(9): 1102-7.
Kersten S, Desvergne B, Wahli W. 2000. Role of PPARs in health and disease. Nature 405: 421-4.
Laakso M, Edelman S V, Brechtel G, Baron A D. 1990. Decreased effect of insulin to stimulate skeletal muscle blood flow in obese man. Journal of Clinical Investigation 85: 1844-52.
Laakso M, Edelman S V, Brechtel G, Baron A D. 1992. Impaired insulin-mediated skeletal muscle blood flow in patients with NIDDM. Diabetes 41: 1076-83.
Law C M, Barker D J, Bull A R, Osmond C. 1991. Maternal and foetal influences on blood pressure. Archives of Diseases in Childhood 66: 1291-95.
Lewis R. M., Vickers M. H., Batchelor D C, Bassett N S, Johnston B M, Skinner S J M. 1999. Effects of maternal captopril treatment on growth, blood glucose and plasma insulin in the fetal spontaneously hypertensive rat. Reproductive and Fertility Development 11: 403-408.
Maffei M, Halaas J, Ravussin E, Pratley R E, Lee G H, Zhang Y, Fei H, Kim S, Lallone R, Ranganathan S. 1995. Leptin levels in human and rodent: measurement of plasma leptin and ob RNA in obese and weight-reduced subjects. Nature Medicine; 1(11):1155-61.
Matsuzawa Y, Shimomurai, Nakamura T, Keno Y, Tokunaga K. 1993. Pathophysiology and pathogenesis of visceral fat obesity. Annals of the New York Academy of Sciences 676:270-8
McNally P G, Lawrence I G, Watt P A C, Hillier C, Burden A C, Thurston H. 1995. The effect of insulin on the vascular reactivity of isolated resistance arteries taken from healthy volunteers. Diabetologia 38: 467-73.
Moller N, Jorgensen A O L, Abildgard L et al. 1991. Effects of GH on glucose metabolism. Hormone Research; 36 (Suppl 1): 32-5.
Nolan J J, Ludvik B, Beerdsen P, Joyce M, Olefsky J. 1994. Improvement in glucose tolerance and insulin resistance in obese subjects treated with troglitazone. New England Journal of Medicine 331: 1188-93.
Randle P J, Garland P B, Hales C N, Newsholme E A. 1963. The glucose fatty acid cycle. Its role in insulin sensitivity and the metabolic disturbances of diabetes mellitus. The Lancet i: 785-789.
Ranke M B, Lindberg A. 1996. Approach to predicting the growth response during growth hormone treatment. Acta Paediatr 85 [Suppl 417]: 18-26.
Rasmussen D D, Boldt B M, Wilkinson C W, Yellon S M, Matsumoto A M. 1999. Daily melatonin administration at middle age suppresses male rat visceral fat, plasma leptin, and plasma insulin to youthful levels. Endocrinology 140(2): 1009-12.
Reaven G, Chang H, Hoffman B B. 1988. Additive hypoglycemic effects of drugs that modify free-fatty acid metabolism by different mechanisms in rats with streptozocin-induced diabetes. Diabetes 37: 28-32.
Rosenfeld R G, Attie K M, Frane J et al. 1998. Growth hormone therapy of Tumer's syndrome: beneficial effect on adult height. J Pediatr 132: 319-24.
Soliman A T, Hassen A E, Aref M K, Hintz R L, Rosenfeld R G, Rogol A D. 1986. Serum insulin-like growth factors I and II concentrations and growth hormone and insulin responses to arginine infusion in children with protein-energy malnutrition before and after nutritional rehabilitation. Pediatric Research 20: 1122-30.
Sonksen et al. 1993. Changes in carbohydrate tolerance in acromegaly with progress of the disease and in response to treatment. Horm Res 40: 68-79.
Steinberg H O, Brechtel G, Johnson A, Fineberg N, Baron A D. 1994. Insulin-mediated skeletal muscle vasodilation is nitric oxide dependent: a novel action of insulin to increase nitric oxide release. Journal of Clinical Investigation 94: 1172-9.
Sugimoto et al. 1999. Pharmacological treatments for GH-induced insulin resistance. Endocr J 46 Suppl:S51-3.
Sugimoto M, Takeda N, Nakashima K, Okumura S, Takami K, Yoshino K, Hattori J, Ishimori M, Takami R, Sasaki A, Yasuda K. 1998. Effect of troglitazone on hepatic and peripheral insulin resistance induced by growth hormone excess in rats. Metabolism 47: 783-7.
Towns R et al. 1994. Pioglitazone inhibits the diabetogenic action of growth hormone, but not its ability to promote growth. Endocrinology 134(2):608-13.
Van den Brandt J, Kovacs P, Kloting I. 2000. Metabolic features in disease-resistant as well as in spontaneously hypertensive rats and newly established obese Wistar Ottawa Karlsburg inbred rats. International Journal of Obesity and Related Metabolic Disorders 24(12):1618-22.
Vickers M, Breier B H, Cutfield W S, Hofman P L, Gluckman P D. 2000. Fetal origins of hyperphagia and hypertension and postnatal amplification by hypercaloric nutrition. American Journal of Physiology (Endocrinology and Metabolism) 279(1): E83-87.
Wade C E, Ortiz R M, Baer L A. 2000. Increases in body mass of rats during spaceflight: models and measurements. Aviation and Space Environmental Medicine; 71(11): 1126-30.
Walker A B, Naderali E K, Chattington P D, Buckingham R E, Williams G. 1998. Differential vasoactive effects of the insulin sensitisers rosiglitazone (BRL 49653) and troglitazone on human small arteries in vitro. Diabetes 47: 810-14.
Walker et al. 1989. Growth hormone treatment of children with short stature increases insulin secretion but does not impair glucose disposal. J Clin Endocrinol Metab 69: 253-8.
Woodall S M, Breier B H, Johnson B M, Gluckman P D. 1996. A model of intrauterine growth retardation caused by chronic maternal undernutrition in the rat: effects on the somatotropic axis and post-natal growth. J Endocrinology 150(2):231-42.

### Industrial Applicability

Compositions of growth hormone and insulin sensitisers can be used in the health care industries to treat undesirable side effects of conventional growth hormone treatment in animals that are small for their gestational age. Methods of this invention can be used to treat adverse side effects, including insulin resistance, that are a consequence of growth hormone therapy.

1. A method of preventing and/or treating an adverse consequence of growth hormone treatment in a juvenile suffering from a growth disorder, the method comprising administering an effective amount of growth hormone in combination with an insulin sensitiser.

2. The method of claim 1 wherein the adverse consequence of growth hormone treatment is insulin resistance.

3. The method of claim 1 wherein the adverse consequence of growth homone treatment is secondary hyperinsulinemia.

4. The method of claim 1 wherein the adverse consequence is selected from the group comprising: a development of diabetes mellitus, dyslipidermia, hypertension and/or obesity.

5. A method of treating a growth disorder in a juvenile comprising administering an effective amount of an insulin sensitiser in combination with the growth hormone treatment.

6. The method of either one of claims 1 or 5 wherein the juvenile is human.

7. The method of either one of claims 1 or 5 wherein the growth disorder is characterised by pre-existing insulin sensitivity.

8. The method of either one of claims 1 or 5 wherein the insulin sensitiser is selected from the group consisting of biguanides and thiazolidinediones.

9. The method of either one of claims 1 or 5 wherein GH is administered in combination with a biguanide insulin sensitiser and a thiazolidinedione insulin sensitizer.

10. The method of either of one of claims 1 or 5 wherein the GH is administered daily by subcutaneous injection.

11. The method of either one of claims 1 or 5 wherein said insulin sensitiser is administered orally.

12. A composition for treating growth disorders and/or preventing or treating the adverse consequences of growth hormone treatment comprising GH and an insulin sensitiser.

13. The composition according to claim 12 further comprising a suitable pharmaceutical carrier and/or excipient.

14. The use of growth hormone and an insulin sensitiser in the preparation of a medicament for treating a growth disorder.

15. The medicament according to claim 14 wherein said growth hormone and said insulin sensitiser in said medicament are adapted for sequential administration.

16. A composition or medicament according to either one of claims wherein the insulin sensitiser is a biguanides, a thiazolidinediones, or a biguanides and a thiazolidinedione.

17. A method of treatment or prophylaxis substantially as herein described with reference to any of the Figures.

18. A composition or medicament substantially as herein described with reference to any one of the examples.

19. A method of treating insulin resistance associated with growth hormone treatment in a juvenile suffering from a growth disorder, comprising administering an effective amount of growth hormone and an insulin sensitiser.

20. The method of claim 19, wherein said insulin sensitizer is troglitazone or metaformin.

21. The use of growth hormone and an insulin sensitiser in the preparation of a medicament for treating an adverse consequence of growth hormone treatment.

22. The method of any of claims 1 or 5, further comprising administering at least one additional insulin sensitiser.

23. The composition of any of claims 12, 14 or 19, further comprising at least one additional insulin sensitiser.
